(19) United States
(12) Patent Application Publication
(10) Pub. No.: US 2014/0228898 A1
(43) Pub. Date: Aug. 14, 2014

(54) UNIVERSAL METHOD AND APPARATUS FOR REPAIRING BONE, LIGAMENT AND TENDON

(71) Applicant: Leonard Gordon, Mill Valley, CA (US)

(72) Inventor: Leonard Gordon, Mill Valley, CA (US)

(21) Appl. No.: 14/099,784

(22) Filed: Dec. 6, 2013

Related U.S. Application Data
(60) Provisional application No. 61/734,371, filed on Dec. 6, 2012.
(60) Provisional application No. 61/763,939, filed on Feb. 12, 2013.
(60) Provisional application No. 61/800,480, filed on Mar. 15, 2013.
(60) Provisional application No. 61/882,577, filed on Sep. 25, 2013.
(60) Provisional application No. 61/899,201, filed on Nov. 2, 2013.

(51) Int. Cl. A61B 17/84

(52) U.S. Cl. CPC A61B 17/84
USPC 606/328

(57) ABSTRACT

A universal method for the repair of bone, ligament and tendon comprises the use of combinations of stabilizers, crimps and optionally anchors to position, provide compression and to promote healing.

### CROSS-REFERENCE TO RELATED APPLICATIONS

This application claims priority to provisional patent Application No. 61/734,371 filed Dec. 6, 2012, provisional patent Application No. 61/763,939 filed Feb. 12, 2103, provisional patent Application No. 61/800,480 filed Mar. 15, 2013, provisional patent Application No. 61/882,577 filed Sep. 25, 2013 and provisional patent Application No. 61/899,201 filed Nov. 2, 2013, the disclosures of each of which are hereby incorporated herein fully by reference.

### FIELD OF THE INVENTION

This invention relates to the repair of bone, ligament and tendon.

### BACKGROUND OF THE INVENTION

The repair of fractures of bone is currently performed using screws, plates or an anchor placed across the fracture, placed in bone fragments or between bones to be aligned. Anchors are used in bone and are attached to sutures which may be placed through a ligament and then tied by a knot. Solid wires, called Kirschner wires or K-wires may be placed between bones or bone fragments. The plates and screws or solid wires are all rigid and solid structures. They all have certain disadvantages including a limited ability to apply compression across a fracture or separated bones, they cannot all be placed in a curved bone and they cannot provide for movement between bones. Screws with variable threads proximal and distal are used to apply some compression as fixation is achieved. The amount of compression is limited and the fixation is limited to compression provided by the screw and threads. Solid wires can migrate causing irritation to soft tissues, may need to be removed surgically if cut subcutaneously and may lead to infection of left protruding through the skin.

A distinct problem in the art is the lack of ability to perform repairs that apply compression to bony fractures, tendon bone connections and ligament bone connections during healing. At present, screws are mainly used to apply compression through the use of multipitched screws and, or in combination with, compression plates. Compression plates use the shape of the screw hole and the seating of the screw head into the compression plate to move bony fragments together and apply some amount of compression.

The present invention addresses and solves this problem through the use of stabilizers that can be applied to repairs under tension and thereby provide greater compression than is possible using screws This invention allows this application to repair small or large bones or comminuted fractures which have many pieces and in which screws cannot be used.

Many methods are used to connect tendons or ligaments to bone. These include various configurations of anchors which have sutures attached. The anchors are fixed in the bone and these sutures are used to tie to the tendon or ligament with knots. The strength is limited by the pullout strength of the anchor and the strength of attachment of suture to the tendon. In one embodiment the tendon or ligament can be placed in bone using an interference method capturing the tendon or ligament between the anchor and the bone. In the present invention crimps are used to hold the sutures and thereby the tendon or ligament on or in the bone.

Scapho-Lunate ligament injuries in the wrist are common and there are currently many methods in use for repair of this ligament. One of the problems present in all methods is the rotational motion of the Scaphoid bone relative to the Lunate bone when using the wrist and hand. As the wrist is brought from ulnar deviation to radial deviation the Scaphoid flexes in position and rotates relative to the Lunate bone. This rotation applies forces across the Scapho-Lunate ligament.

For acute injuries, direct repair of the ligament and pinning is possible if adequate ligament remains to do so. This direct repair and pinning may be combined with supplemental tendon procedures such as the Blatt or Brunelli procedures in an attempt to hold the bones in position. Because of the forces applied across the ligament in this joint no procedure is universally successful. Screws placed across the bones such as in the Razzle procedure have had some success but with a high incidence of screw breakage over time.

Other methods use tendons attached to a suture anchor within the Lunate in an attempt to connect the bones (Arthrex method). However, the poor quality and strength of tendons and allografts combined with the problems with placement of anchors in the Lunate make these methods difficult and complex to do so that successful results have not been confirmed.

The current invention describes a simple method using multi filament stainless steel cable which can rotate and flex without compromising its strength. This is combined with a method of attachment to the bone on both sides which is extremely strong and simple to accomplish.

Many methods are currently in use for the repair of large tendons such as the Achilles tendons. Most of these require extensile surgical exposure to dissect and connect the proximal and distal ends of the Achilles. For example the Krakow stitch is often employed which is a time consuming, complex procedure that requires wide exposure and a long incision in order to accomplish the repair. This repair is then completed by attaching Fiber Wire or other sutures with a knot at the repair site. Other methods, such as the Tenolig, use smaller incisions at the surgical site but the repair lacks strength and reproducibility. The present invention provides methods of repairing the Achilles with smaller incisions, reduced complexity and increased speed.

Another common sports injury is the avulsion of the Biceps from the radial tuberosity. There are many methods in current use for reattachment of this tendon. These methods fall into two categories, a single incision volar approach and double incision methods. In the volar approach method, bone anchors are used to attach the Biceps tendon to the bone. The bone anchors are difficult to position in the bone and the attachment is not as strong as is needed for early Range of Motion rehabilitation protocols.

The double incision methods utilize attachment of the tendon to the Biceps tuberosity and sutures brought out through the bone cortex and tied with a knot through a separate dorsal incision.

The present invention addresses these problems by providing a single incision procedure that allows attachment of the tendon with multi filament stainless steel cable which is brought out of the volar cortex of the radius bone 1-2 cm from the radial tuberosity and attached by a crimp and washer to the volar cortex of the radius thereby securing the tendon. This is much simpler and stronger than use of a bone suture anchor.

Fractures of the volar portion of the middle phalanx at the Proximal Interphalangeal (PIP) joint of the finger can be difficult to repair due to the small size of the bone fragments which are attached to the volar plate and ligaments of the joint.

These PIP joint fracture dislocations are difficult to reduce and often have serious long term functional loss of range of motion and arthritis due to the position of the middle phalanx. When the volar portion of the middle phalanx is fractured and the volar fragment is large enough to include the collateral ligament attachment then the dorsal fragment migrates and dislocates dorsally as shown in FIG. 19A. This is an extremely difficult problem to correct and results are often poor.

Many methods have been employed to correct and hold the volar fragment in position to allow it to heal. These include external fixation with pins placed in the proximal and middle phalanges creating direction of forces to re-locate and hold the reduction (Agee method). Other methods include internal fixation with pins or screws and suture anchors. These are difficult to manage and because of the small size of the volar fragment usually do not hold adequately. The present invention advantageously addresses these difficulties by providing a methods of quickly and stably positioning the fragments and correcting the dorsal position of the middle phalanx dorsal fragment.

### SUMMARY OF THE INVENTION

The invention provides a universal method for repairing an anatomical member, such as a bone, a tendon or a ligament. In the case of ligaments, the method can be used to align separations such as multiple bone fragments so the bone fragments can heal and multiple bones so that the ligament can heal or be repaired. The method employs repair devices that provide structural means to secure opposed ends of the anatomical members and in appropriate repairs, provides a novel method of applying distraction or compression during reduction and healing. In this invention a novel method of attachment of the wire or cable to the bone using crimps is employed in order to provide distraction or compression to achieve the desired proximity across the junction. This junction may be a fracture, i.e.: two parts of the same bone, or a ligament i.e.: two bones that need to be connected. In the case of a tendon bone connection the junction is a tendon and bone secured by a crimp.

### DESCRIPTION OF THE DRAWINGS

FIG. 1A illustrates an example of the universal method of repair of a bone fracture.

FIG. 1B illustrates an example using a cannulated stabilizer

FIG. 1C illustrates an example using a stabilizer with one section with a greater diameter at the leading end and another section with a smaller diameter creating a drill hole form fitting on the distal end and larger than the diameter of the stabilizer on the proximal end. The larger diameter end may have threads as shown.

FIG. 1D illustrates an example using a combination of a cannulated stabilizer and a flexible stabilizer cable.

FIG. 1E illustrates that stabilizers (cannulated or solid wires or flexible cables) may be placed in various configurations.

FIG. 2A illustrates a repair of a bone having multiple fractures.

FIG. 2B illustrates an exemplary reduction plate.

FIG. 2C illustrates an exemplary reduction plate.

FIG. 3 illustrates some of the various designs of crimps that may be used in the present invention.

FIG. 4 illustrates examples of anchors.

FIG. 5 illustrates examples of anchors and crimps applied to a stabilizer cable.

FIG. 6 illustrates a solid wire (above) or a cable or suture (below) is passed from one bone to another and held in place at one end with a crimp and at the other end by a second crimp.

FIG. 7 illustrates a solid wire (above) or a cable or suture (below) is passed from one bone to another and held in place at one end with an anchor and at the other end by a crimp. The crimp is applied after traction on the wire thereby compressing the bones together.

FIG. 8 illustrates that one end the cable or suture is attached to the tendon (or ligament) and then passed through the bone and a crimp placed on the far side of the bone allowing traction and secure attachment to bone.

FIG. 9A illustrates a curved drill guide to make drill hole in bones to be connected. May be of variable shape and size depending on curvature and size of bones.

FIG. 9B illustrates a more detailed view of a straight drill guide with a second curved drill guide within the straight guide drilled within the Scaphoid bone poised to drill a curved passage for a guide-wire to be drilled into the Lunate bone.

FIG. 10 illustrates a drill bit end to make curved hole.

FIG. 11 illustrates a tube or sleeve can be placed in drill hole to replace drill guide.

FIG. 12 illustrates a stabilizer such as a multi filament stainless steel cable or other suture with or without specific coating placed within the drill guide or sleeve from the first bone to the second one and into the core of the second bone. The coating of the cable or suture is shown.

FIG. 13 illustrates that after engagement of the far end in the second bone, traction applied to the cable or suture bringing the bones or two parts of the same bone together and a crimp is applied at the proximal end of the first bone to fix the tension and hold the two bones approximated.

FIG. 14 illustrates examples of methods of repairing a rupture of the Achilles tendon (A-D) and an example of reattaching the Achilles tendon to the Calcaneus bone (E).

FIG. 15 illustrates another example of repair of of the Scapho-Lunate.

FIG. 16 illustrates another example of repairing the Scapho-Lunate using trabecular metal coating or hydroxyappetite.

FIG. 17 illustrates a Scapho-Lunate reduction clamp.

FIG. 18 illustrates an exemplary method of repairing a Biceps tendon be reconnecting the tendon to the radial tuberosity (A-F), a curved suture guide and a washer (G).

FIG. 19 illustrates an exemplary repair of a Proximal Interphlangeal joint fracture,

FIG. 20 illustrates the use of cannulated screws in a Scapho-Lunate repair.

### DETAILED DESCRIPTION

The current invention provides a universal method for repairing bone, tendon and/or ligament. In a general embodiment of this invention, a stabilizer is used to align two parts of a fractured bone or to stabilize and repair an osteotomy or two bones with a ligament or tendon injury. The stabilizer and bone, tendon or ligament(s) are held in place using crimps or a combination of a crimp and an anchor attached to the stabilizer. Unlike prior uses of crimps and/or anchors with wires or sutures, the present method comprises placing the stabilizer internally in the bone. Tension can then be applied to the stabilizer before the attachment of the crimp. This advance provides longitudinal compression of the site of repair along the path of the stabilizer through the site of the repair. The compression achieved using the stabilizer crimp method described herein is greater than previously possible using sutures, screws or externally placed wires.

Separations, as used herein, can be any type of separation between anatomical features of bone, ligament and tendon. For example, separations include fractures across bones, torn ligaments, ruptured tendons and the like. The method can be used to apply compression, distraction or both across a separation. Compression can be applied in instances of bone fractures and the reattachment of tendons to bone where a desired proximity is contact between the anatomical features. Distraction, compression, or both can be applied in cases of ligament separation to achieve the desired proximity between the bones to which the ligament attaches.

The stabilizer may be either a flexible cable of multi filament stainless steel (MFSS), malleable and flexible steel or some other flexible cable or substance or it can be a rigid wire, e.g.: a Kirschner or K-wire, or a pin, or a cannulated wire or pin or similar rigid means. Stabilizers may be coated with nylon, a polymer or any other synthetic substance or have a biologic material surrounding or embedded in it. Coating may prevent irritation of bone or tissue. Stabilizers may be porous, sputter coated to have porous surfaces or treated with compounds to promote bone in-growth. Stabilizers may be provided coated by or placed within biological materials such as ligament or tendon. The can also be coated with ETFE (Ethylene tetrafluoroethylene), PFTE (Polytetrafluoroethylene), PEEK (polyether etherketone), Nylon or other polymers or coatings.

The stabilizer is held in place through the use of crimps or a combination of an anchor and a crimp. If it is desirable to have movement between the bones being repaired, such as in ligament repairs, then a stabilizer that is flexible is preferable. If it is desirable to hold the bones together with as little movement as possible, then a rigid stabilizer such as a wire or pin would be appropriate, although cables can also be advantageously employed under tension. In some repairs it may be desirable to have some parts of the repair held without movement and allow other parts of the repair to move in which case a combination of flexible and rigid stabilizers are used. If the cable is subject to torsion or twisting then the design of the cable i.e., the internal configuration or lay must be such as to allow the torsion in the direction appropriate for the lay.

In certain applications of a stabilizer in combination with a crimp, the crimp may move laterally along the stabilizer over time as a result of the tension applied across the stabilizer. Creating a solid bond between the crimp and the pin to fix the crimp in order to maintain position and compression is challenging. Inadequate attachment and movement of the crimp may occur when using solid pins or wires as stabilizers. The movement results in a loss of tension across the length of the stabilizer and a failure of compression across the repair site.

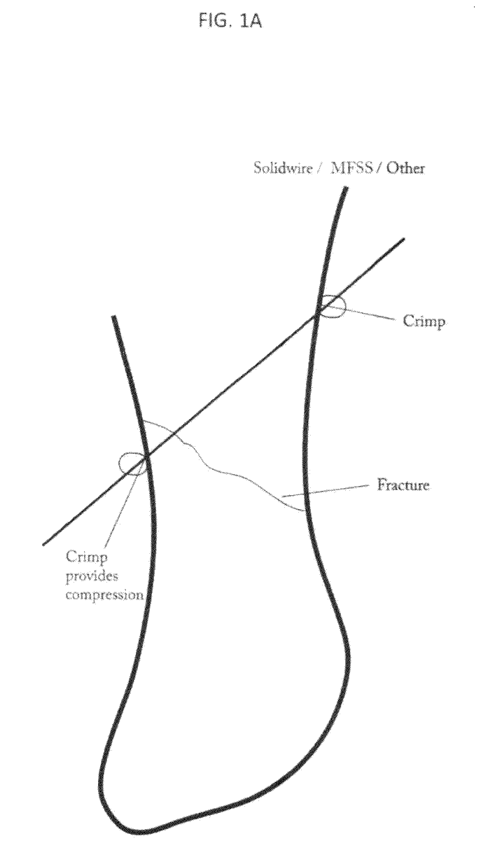

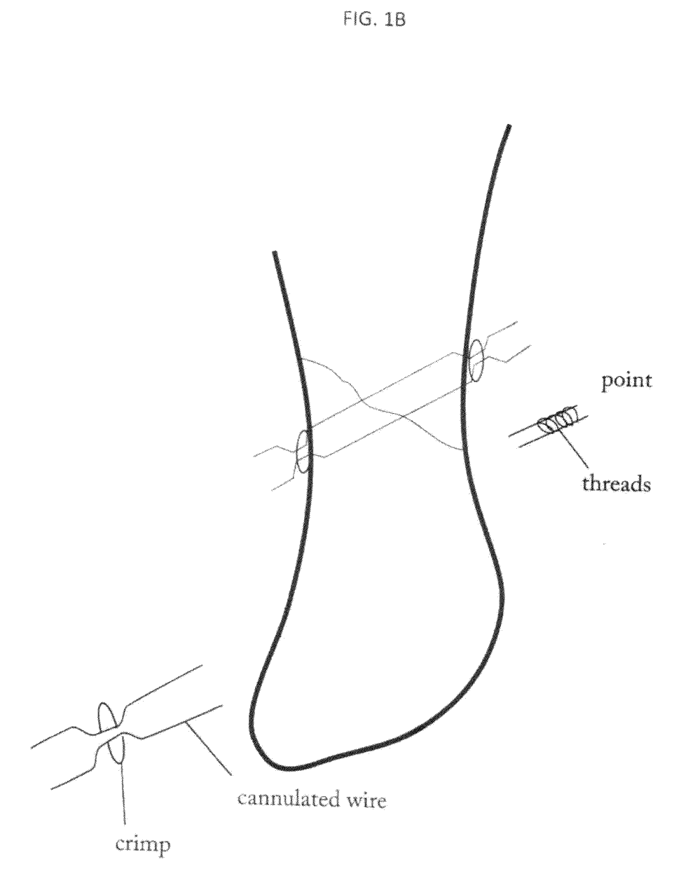

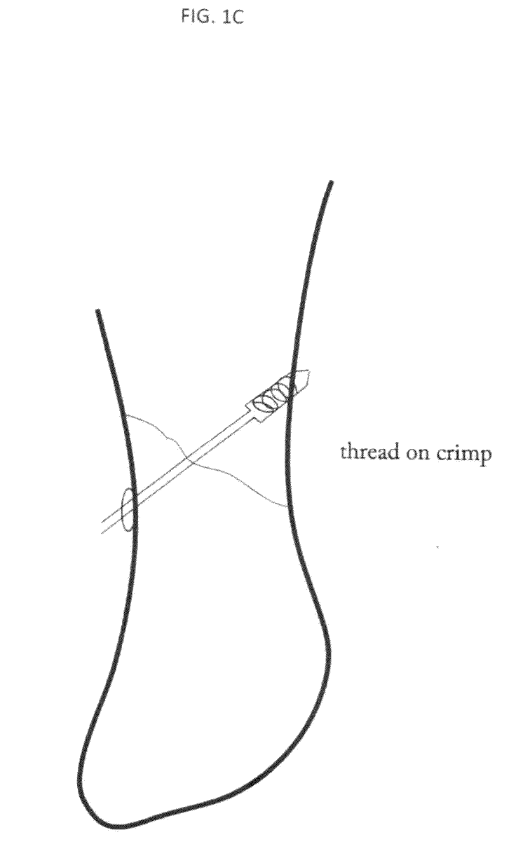

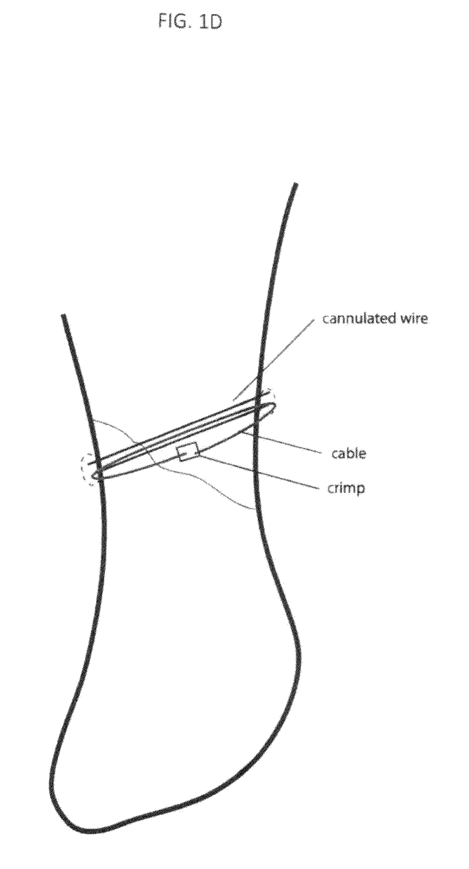

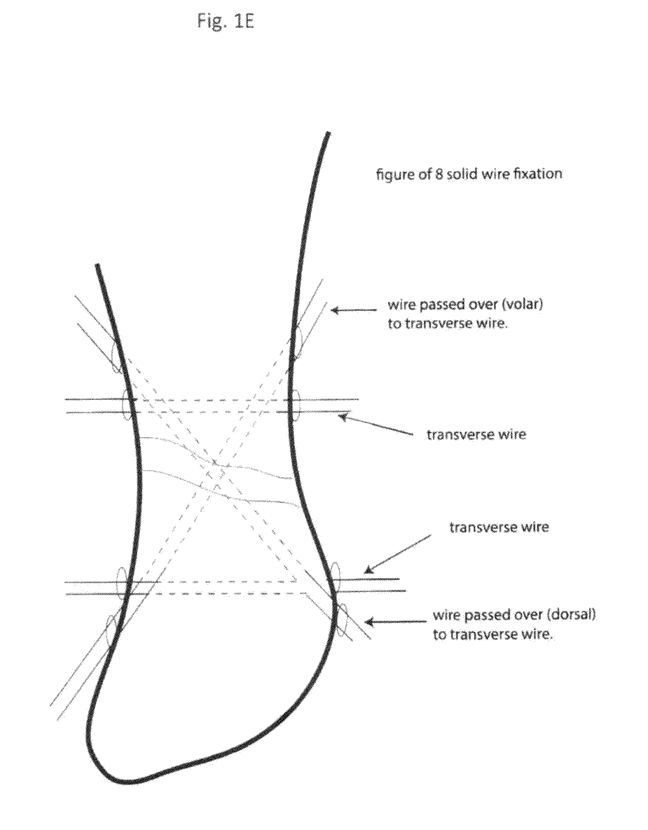

This potential problem is overcome by the present invention by employing a stabilizer that is deformable such as a cannulated wire or pin. The deformable stabilizer is compressible and therefore deformed upon application of the crimp. The deformation of the stabilizer by the crimp prevents the crimp from moving laterally along the stabilizer. Therefore, the deformation of the stabilizer effectively locks the crimp in place and allows a chosen level of compression to be applied to the repair site over long periods of time. An exemplary use of a deformable stabilizer is depicted in FIG. 1B.

Examples of a deformable stabilizer would include a cannulated wire, a cable such as a multifilament stainless steel or polymer cable, sutures including those made of a deformable plastic, polymer, elastic or other suture material. Wires made of malleable, deformable material or other substances that can be deformed or compressed upon application of the crimp. Preferred materials are those that can be deformed by the application of a crimp while retaining the capacity to apply the appropriate compression across the site of the repair.

Stabilizers can be designed and fabricated to incorporate various useful features. For example, a stabilizer can be designed to be broken at specific points, for example, by scoring to weaken the stabilizer. This allows the length of the stabilizer to be adjusted by simply breaking it at a chosen point. A stabilizer may be broken before or after insertion into an anatomical feature and before or after placement of the crimp.

Stabilizers can de designed and fabricated with various ends including points for drilling or rounded ends that will not irritate soft tissue. Stabilizers may have threads for attaching drill bits for drilling into bone, anchors or other accessories. If the stabilizer is cannulated, it may have internal threads. Various other useful features may be employed as will be apparent to one of skill in the art.

In the most general embodiment of the invention applied to repairing a broken bone, a passage is drilled in the bone entering the cortex of the bone, passing across the point of fracture and exiting the cortex of the opposite side of the bone. A stabilizer such as a flexible cable or solid wire is placed in the passage. On one side of the bone a crimp is placed on the stabilizer at the outside cortex of the bone. The crimp is designed to sit against the cortex and sized to remain outside the passage. The fractured bone is secured by applying tension to the stabilizer and placing a crimp on the outside of the cortex of the bone opposite the first crimp. This arrangement and the use of at least one crimp allows the surgeon to apply compression to the repair prior to crimping.

The stabilizer crimp or anchor system of the present invention can also be used to provide distraction between bone fragments. This can be useful when a portion of a bone is lost and appropriate spacing of the remaining bone segments need to be maintained.

The repair methods of the present invention can also be used when an osteotomy is performed.

In another embodiment, a passage or bore is created entering the cortex of the bone, crossing the point of fracture and entering the bone on the other side of the fracture. In this embodiment, the passage does not exit out through the cortex on the opposite side of the bone. In this embodiment, a stabilizer such as a wire or cable with an anchor at the first end of the wire is used. The first end of the wire is placed within the bone at the far end of the passage. The anchor grips the bone to hold the wire in place in the bone. The anchor can be an expansion anchor, a friction fit anchor, a tilting anchor, a screw in anchor, or other anchor known or developed in the art. Some anchors are particularly useful when placed using cannulated devices. For example, anchors that can be placed on the end of a stabilizer and slid within a tube but expand upon being pushed out the far end of the tube are useful for anchoring the distal end of a stabilizer in a bone. The anchor may be manufactured attached to the stabilizer, crimped to the stabilizer, screwed onto the end or otherwise applied to the end of the stabilizer. The anchor may be folded within a tube or cannulated pin like an umbrella and when it reaches the far end of the cannulated pin or tube it opens up and thereby fixes in the bone. Once the anchor is firmly in place, the surgeon can apply compression to the fracture or bones by applying tension to the stabilizer. A crimp is then placed on the stabilizer outside the cortex of the bone to maintain the compression on the fracture or bone.

This type of repair is particularly useful when a fracture is being repaired close to a joint. In these cases a passage is drilled from the exterior cortex of the bone, across the fracture point and into the portion of the bone forming the joint. A first crimp is applied to the far end of the stabilizer at the end of the passage in the bone forming the joint, a stabilizer extends from the crimp across the fracture point to the exterior of the cortex opposite the placement of the first crimp, tension is applied to the stabilizer to compress the bone fragments across the fracture point and the second crimp is attached at the exterior cortex. In this embodiment when the bone fragments are small and a screw is too large a K-wire can be used as described and is placed across the small bone fragment and out of the far cortex. A crimp is placed on one side and traction can be applied to the wire on the other side thereby reducing the small fragment(s) of bone and a crimp can be applied to the second end of the wire or cable to maintain the position. This may be particularly applicable to avulsion ligament injuries or fractures of small pieces of bone such as a dorsal fracture dislocation of the Proximal Interphalangeal joint of the finger.

In another embodiment, the method can also be used to attach a tendon or ligament to bone. In this instance of the method, the tendon or ligament is on one side with the cable or suture placed through it to provide a strong attachment. A passage is created in the bone beginning at the point where the tendon or ligament will be attached, passing through the bone and exiting the cortex opposite the point of entry. The cable or suture is then brought through the passage in the bone and the crimp placed on the cable where it exits the far cortex of the bone. As with bone to bone contact this also allows tension to be applied to the connection between ligament or tendon and the bone. The tension is applied and the crimp placed to hold the cable or suture in position with the tendon or ligament in contact with the bone. In some embodiments of the invention, the passage can be enlarged, e.g., over-drilled, at the point of attachment of the tendon or ligament to the bone and then become a narrow passage for suitable acceptance of the wire, cable or other stabilizer. This allows the tendon or ligament to be drawn into the bone a suitable distance which can enhance the strength of the repair for healing.

An aspect of the invention is the repair of ligaments between bones. The method of the invention is particularly well suited to repair of the Scapho-Lunate ligament, Acromio-Clavicular ligament or collateral ligaments of the thumb Metacarpo-Phalangeal joint and similarly situated ligaments. At present, longitudinal screws, static K-wires or anchors on one or both sides of the injured ligament have been used to repair ligaments such as the Scapho-Lunate ligament. That method of repair is static and rigid and usually does not allow compression so that an external device is needed to reduce the bones into position. If some compression is applied it is through the threads of the screw. Current methods allow for little or no rotation or motion of one bone in relation to another as occurs with the normal kinematics of bones. This is especially noted in the wrist where the Scaphoid flexes and extends as the wrist moves from radial to ulnar deviation. The lack of rotation or motion can lead to breakage of the wires or screws over time. The present invention overcomes these problems by using flexible cable with crimp attachments, or on one side an anchor attachment, rotational motion is provided and the use of a crimp allows longitudinal compression of the two bones or parts. The optimal rotation direction on a cyclic or repetitive basis is governed by the configuration of the internal structure of the multi filament cable.

Another method of the present invention involves passing the stabilizer as a guide wire and drilling over the stabilizer to make a passage for a cannulated screw or tube. The tube may be metal or some other substance. The wire may be passed through said tube from one bone segment to the other out of the end of the far bone or put within the substance of the bone. Said wire may have an anchor or screw attached at the far end or such anchor or screw may be connected between the two bones to be fixed. The second end of the wire may be fixed by applying a crimp.

Another advantage of the present invention over the use of screws is that the stabilizers employed, MFSS and wires, can be much thinner than the screws typically used in bone repair. The thinner wires and cables allow passages to be drilled that are smaller than the holes required by screws. This leads to less shattering of bone and the ability to repair more fragile fragments of bone than is possible when using screws.

Like screws, the stabilizer wires, pins and cables can be used with plates, bone holders and the like. Instead of applying bone holders and other types of plates to the bone with screws, passages are drilled and cables or wires are placed in the passages and extend through holes in the plates. Flexible cables can be woven through passages in the bone and holes in the plate to stitch bone fragments together. Tension is applied to the stabilizers to align and compress the bone fragments to the plate or bone holder and crimps are applied to maintain the compression during healing. Unlike screws many wires can be placed across fractures in multiple directions because of the small holes made in the bone to place these wires. Unlike prior use of cables the cables which are placed outside the bones to hold the position which does not allow for compression or use of wires or anchors in the present embodiment the cables passed within the bones and fixed by crimps so that this allows fixation of comminuted fractures, longitudinal compression using crimps and use of anchors. This method also allows for the attachment of ligaments and tendons also using these crimps.

Another advantage of the use of the stabilizer and crimp system of this invention is that the stabilizer and crimp may be left in place after healing. This is due to the use of crimps and which prevent the stabilizers from migrating. For this reason wires that otherwise must be removed may be left in place.

This method of crimp attachment also allows much greater strength of the attachment of the repair device to the bone. For example, at a first end of a stabilizer there can be a bone anchor and a wire or cable can run through a bone or bone fragment where a crimp is applied to the second end of the stabilizer. When using screws, the strength of the connection is dependent on the strength of the interaction of the screw and the bone. If the bone is fragile, once the screw is placed in the bone the connection will be fragile. Unlike screws, the use of crimps does not rely on the strength of the connection to the bone. The crimp connection derives its strength from the metal to metal connection of crimp to stabilizer wire, pin or cable.

When applied against the exterior cortex of bone, the crimp can be designed to spread the pressure over a larger or smaller area depending on the strength of the underlying bone and the amount of compression being applied to the repair. The crimp can be tubular, round or any other shape and can be counter sunk. The key functions of the crimps are to hold the stabilizer, e.g., MFSS, pin or wire, and does not allow it to loosen or retract into the passage that has been drilled in the bone. The crimp can be designed to be countersunk into the cortex of the bone. When used with cannulated compressible stabilizers, crimps may have gripping ridges, points or other modifications on their internal surfaces to enhance the deformation of the compressed stabilizer. Such modifications can help to prevent the crimp from moving along the stabilizer after application. They can also allow one to trim the excess length of the stabilizer immediately adjacent to the crimp. The crimp can also be designed to have a rounded head to reduce the possibility of irritation of soft tissues.

In some instances a backing plate or washer can be inserted between the crimp and the bone to spread the pressure applied to the bone cortex when applying a crimp to a wire or cable under tension. A backing plate can also be useful in instances where a number of small bone fragments adjacent to each other are to be repaired. The plate can be use to hold the small pieces in position.

The use of multifilament stainless steel wire (MFSS) as the stabilizer is particularly useful in the present invention. The MFSS can be placed in a curved or straight hole drilled into the bone fragments in order to apply compression and repair and fix fractures of bones in any configuration i.e.: longitudinal, angled, circular or combined. MFSS can be used to hold bones in position to replace the function of ligaments and allow healing of ligaments between properly positioned bones. Under sufficient tension, MFSS can provide stability comparable to a solid wire. In other applications, MFSS can provide movement such as the rotational motion desirable in the instance of a Scapho-Lunate ligament repair.

The invention will now be described with reference to the particular embodiments shown in the figures. FIG. 1A shows a wire, cable, pin or other stabilizer placed through a bone fracture and held in place with crimps. If one intends that the bone move as little as possible, then a solid wire or pin would be an appropriate choice of a stabilizer. The stabilizer extends from the exterior cortex of one side of the bone, through the interior of the bone, across the point of fracture and exits the exterior cortex of the bone opposite the point of entry. A crimp is placed on the stabilizer adjacent to the exterior cortex of one side of the bone, tension is applied to the stabilizer to compress the fracture together and a second crimp is placed on the means and against the exterior cortex at the opposite side of the bone from the first crimp. After application of the crimps, the stabilizer may be trimmed flush with the crimps. In this figure, the placement of a single stabilizer is shown. However, a surgeon may place multiple stabilizers as needed to compress and stabilize the fracture to the degree desired by the surgeon in his judgment. FIG. 1B shows the placement of a cannulated stabilizer.

Compression across a fracture or across two bones is generally provided by using a screw with different threads in the proximal and distal aspects so the bone fragments are brought together. Alternatively a screw with a ‘head’ is used and the distal threads of the screw are used to engage the far cortex. The near or proximal cortex is over-drilled creating a wider drill hole so the screw can glide in this hole. As the screw is engaged in the far bone the head of the screw acts to compress the fracture. This requires over drilling of the proximal drill hole. In the present invention the diameter of the wire is used to create a larger hole and also engage the distal cortex. A crimp is used to then crimp the wire at the proximal end and thereby maintain the compression. There is currently no method of providing compression for wires without over drilling the proximal hole. The present invention describes a method of compression using this wire (solid or cannulated) combined with the method of crimping the wire, using a cannulated wire

FIG. 1C shows a stabilizer which may be solid or cannulated wire with one end of a certain diameter and which may be threaded at the end and may have a point. This end may be drilled across the repair site and engaged in the distal aspect or cortex of the bone. Alternatively, the bone maybe drilled first before placement of the stabilizer to engage the distal aspect or cortex of the bone. The near or proximal end is narrower so it glides in the bone. Once traction is applied on the stabilizer, the distal bone is pulled closer thereby compressing the fracture or bones and bringing them closer together. The proximal end is then crimped to hold this position.

In another embodiment similar to that shown in FIG. 1C, a stabilizer is fitted at a first end with a point or drill bit. The first end is employed to attach to the far cortex or it may pass through the bone across the repair site and exit the far cortex. Once the first end has exited the point or bit can be removed, in certain embodiments, by snapping it off at a preselected weakened point such as a pre-scored point on the stabilizer or in another embodiment it can be cut off. The first end may then be crimped adjacent the far cortex of the bone, traction applied across the stabilizer and with a the near pin having a smaller diameter than the hole traction may be applied and a crimp placed on the stabilizer adjacent the proximal cortex of the bone. The length of the stabilizer may then be adjusted by snapping off the excess proximal portion of the stabilizer at a pre-scored point or cutting it off.

A feature that may be included in this type of stabilizer is an umbrella like expanding disk attached to the shaft of the stabilizer. It is placed adjacent to the preselected weakened point at the end of the stabilizer that has the larger diameter. In embodiments where the larger end is bored completely through the bone and out the distal side, the larger end may be removed by cutting or snapping the stabilizer at a preselected weakened point. The cut or weak point is between the larger end and the umbrella like expanding disk. The disk is pushed through the distal exit of the bore, expands and is pulled back against the cortex of the bone. Traction is then applied and the stabilizer is crimped adjacent the proximal entrance of the bore.

FIG. 1D shows the use of a cannulated pin stabilizer in combination with a flexible cable stabilizer. In this embodiment, the rigid stabilizer is placed within the bone. The stabilizer usually will cross the repair site but it is not required in this case. In some cases, it may be preferable that the stabilizer does not cross the repair site. Once in place, a flexible cable stabilizer such as a multi filament stainless steel cable is placed within the cannulated pin stabilizer. The ends of the cable protruding from each end of the cannulated pin are wrapped around the bone to hold the pieces in the desired position for repair. Traction is applied to the cable and the ends are then crimped together and trimmed. The cable can be placed without a crimp on the bone surface or passed around the bone after the crimp on the bone surface is used to compress and fix the cannulated pin.

Stabilizers, e.g., wires that are cannulated or solid or flexible cables may be placed in various configurations as shown in FIG. 1E. One configuration, for example, could be a figure of 8 tension band using the cannulated wire with the proximal or distal longitudinal wires placed dorsal to a transverse wire and the wire at the opposite end traversing volar to the opposite transverse wire, thereby creating a tension band or frame that resists flexion forces.

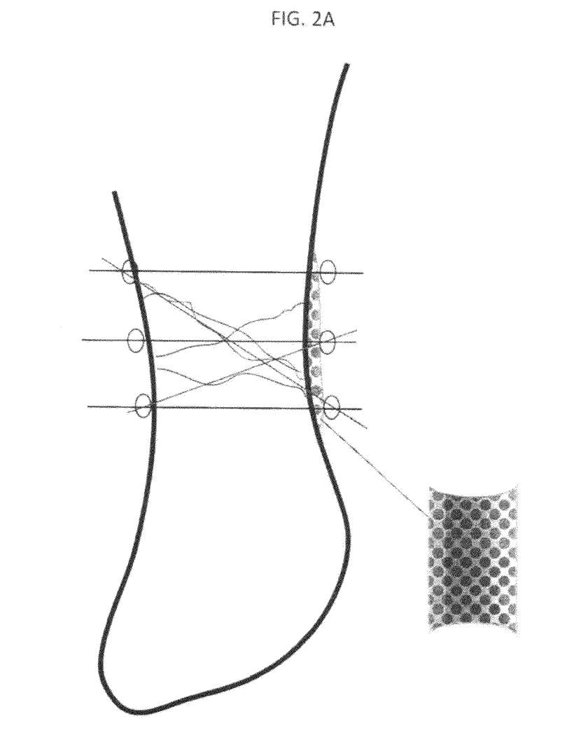

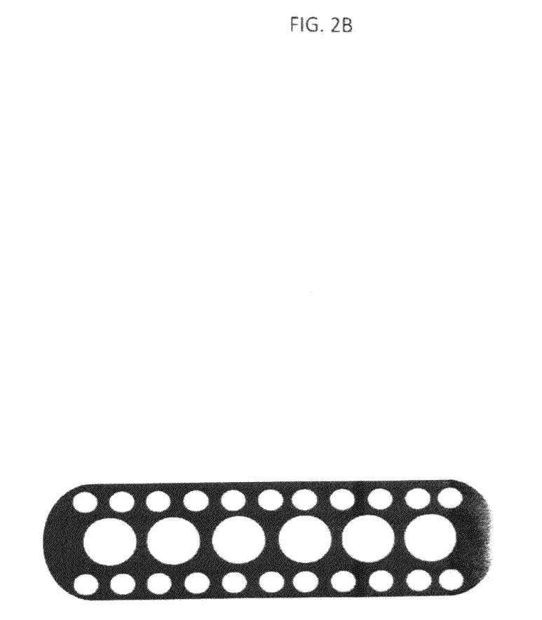

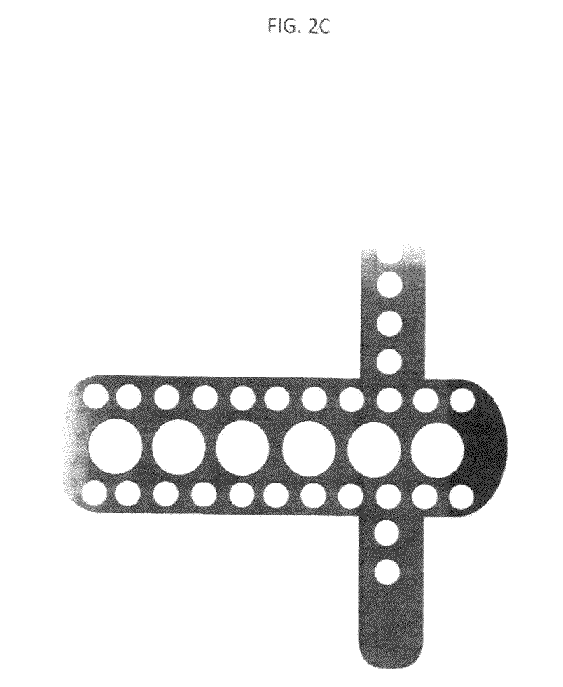

The exemplary placement of multiple stabilizers is shown in FIG. 2A. In this Figure, there are multiple fractures shown in the bone presenting a more difficult repair scenario. The repair shown uses multiple stabilizers in conjunction with a plate to provide additional stability shown along the right side of the bone. The crimps are placed on the stabilizers against the exterior cortex on the left side of the bone and against the plate on the right side of the bone. The combination of the compression provided by crimping the stabilizers under tension and the plate holds the multiple pieces of fractured bone securely during healing. The plate may be a solid sheet cut to size through which the stabilizer may be drilled, and the crimp placed on the outside of the plate holding the stabilizer and bone in place.

In certain embodiments of the invention, the reduction plate is not left in the body. Instead, the reduction plate is used only to position the stabilizers. It is placed on the end of a stabilizer i.e., the stabilizer is placed through one of the larger or smaller holes on one side of the fracture i.e., proximal or distal. A second or third stabilizer is placed through one of the holes in another part of the bone, frequently on the opposite side of the fracture. By selecting the hole to be used the bone may be straightened and the position or angle of the bone changed. Because of the variety of holes, wires can passed through in whatever configuration necessary, thereby correcting any deformity in the longitudinal plane. The crimps are then placed holding the stabilizers in position. The reduction plate is then removed i.e., it is not actually fixed or connected. The reduction plate can be placed on one or both sides of the fracture or bone for ideal positioning and once the position is fixed the plate(s) are removed. FIG. 2B shows an exemplary reduction plate.

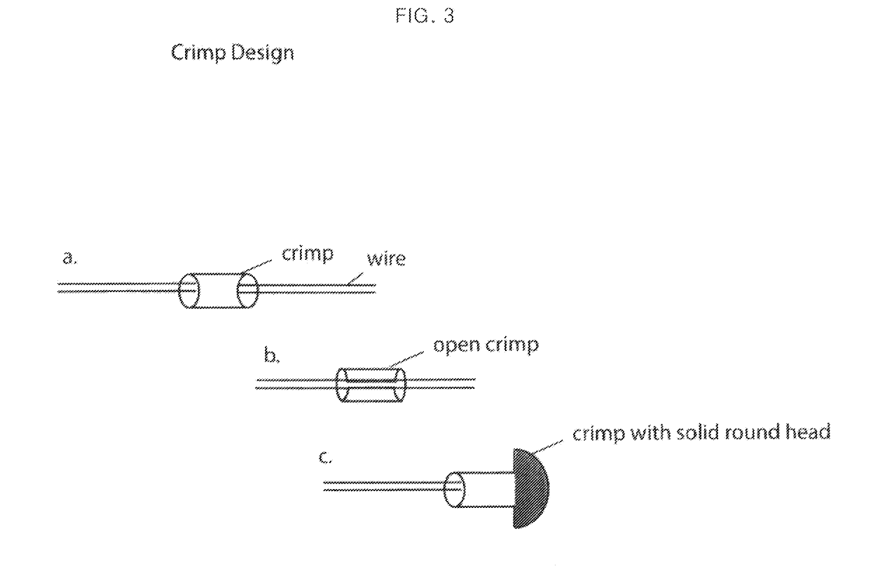

For reduction of rotatory mal position the proximal or distal segment may be rotated to correct any deformity and the wires passed through the holes in the plate within flanges so as to correct and hold the rotatory position while the crimps or plates are placed on the bone to maintain the reduction. Another exemplary plate with flanges is shown in FIG. 2C. As above, the plate may be removed rather than left in the body. FIG. 3 shows some of the various crimps forms that can be employed in the present invention. At the top. A crimp is used that is slid over the end of the wire or other stabilizer. In the middle, a crimp has a lateral opening that allows one to place it on the stabilizer at any point. At the bottom, a crimp is shown with a rounded top to reduce the possibility of irritation of soft tissues. This crimp also has a stem below the head where it can be crimped onto a stabilizer. The stem is meant to be countersunk into the bone which may be accomplished by over-drilling the entrance of the passage to accommodate the stem. Other crimps may also be used in the invention. Crimp may be of variable configuration and shapes comprising a lumen throughout or a cap made of a different material from the stalk with only the stalk being compressed for attachment to the wire. Their design is limited only by the fact that the crimp must be able to securely hold the stabilizer in place. When used under pressure to compress a repair, the crimp must be able to maintain an integral connection with the stabilizer.

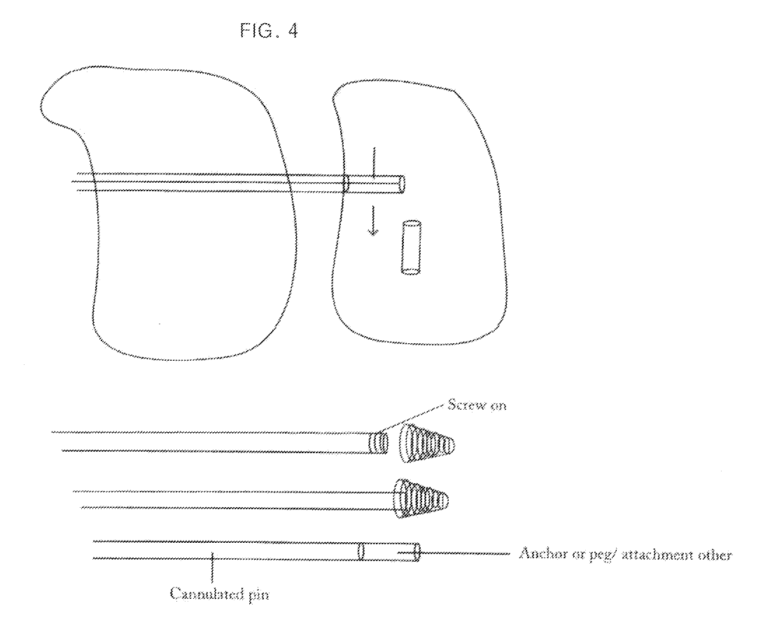

FIG. 4 shows some examples of anchors that may be used with the present invention. At the bottom of the figure there is shown a screw on anchor that can be applied to the end of a stabilizer, then it is shown attached to the stabilizer. Below that is shown a cannulated pin with an end that can accept an anchor within the pin. In the top of the figure a stabilizer is show extending through one bone and into another bone. In this case, a passage can be drilled through the first bone and the stabilizer inserted through it without the anchor attached. Then the anchor is applied to the stabilizer and inserted into a passage in the second bone. The anchor and cable can be aligned within the tube and once the cable and anchor are advanced into the far bone the anchor rotates so with removal of the tube the anchor now lies at an angle, ideally a right angle, so as to be fixed into the far bone.

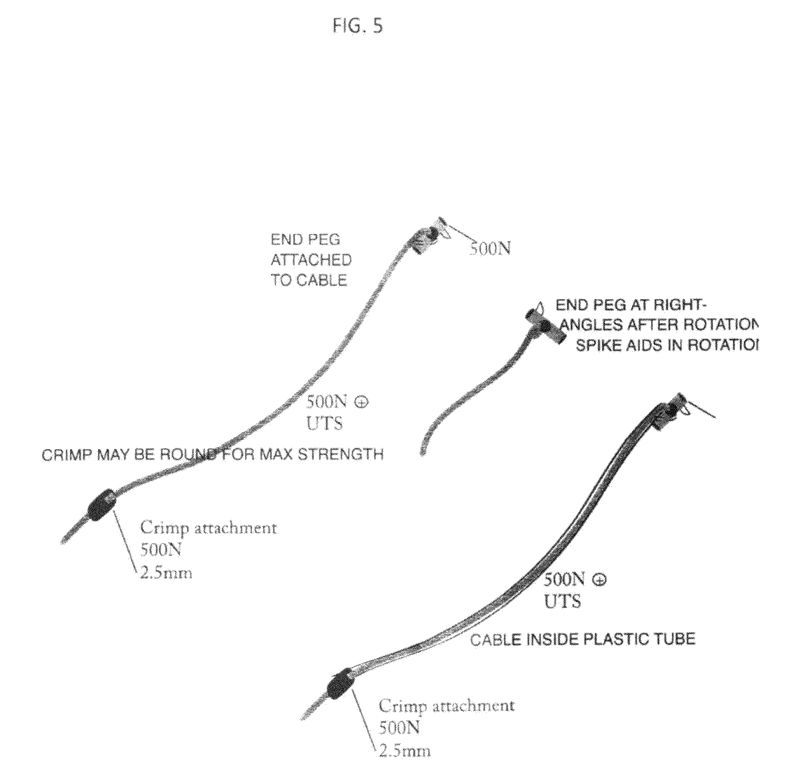

FIG. 5 illustrates a peg anchor attached to the end of a stabilizer cable. In one embodiment, the peg end is passed all the way through a passage through one or more bones or bone fragments and exits the far side. The connection of the stabilizer to the middle of the peg allows the peg to turn and adopt a conformation that prevents the peg from reentering the passage. Tension is then applied to the opposite end of the stabilizer and a crimp is applied to hold the repair in place during healing. Alternatively, a solid wire with an anchor on the far end can be passed into the far bone. The anchor can be of variable configuration and design. The solid wire is within the near bone and traction applied to position the bones with compression and an anchor is placed to hold the wire in position.

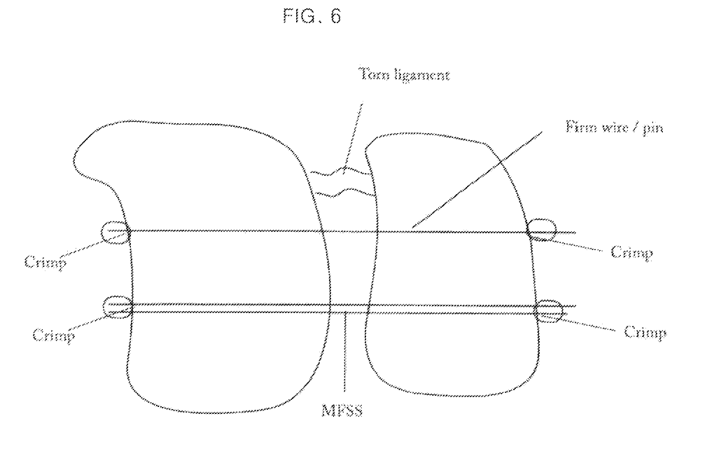

FIG. 6 shows the repair of a ligament injury between two bones using the method of this invention. In this figure, a rigid stabilizer is employed in a passage through both of the bones in proximity to the damaged ligament. The stabilizer is crimped in place to hold the two bones in the correct alignment for the promotion of healing of the tendon. The two bones are may be alternatively stabilized by employing a flexible stabilizer at a location distal to the damaged ligament. The use of a flexible stabilizer at the distal location allows for some movement of the bones in a manner that preserves some of the natural movement between the bones.

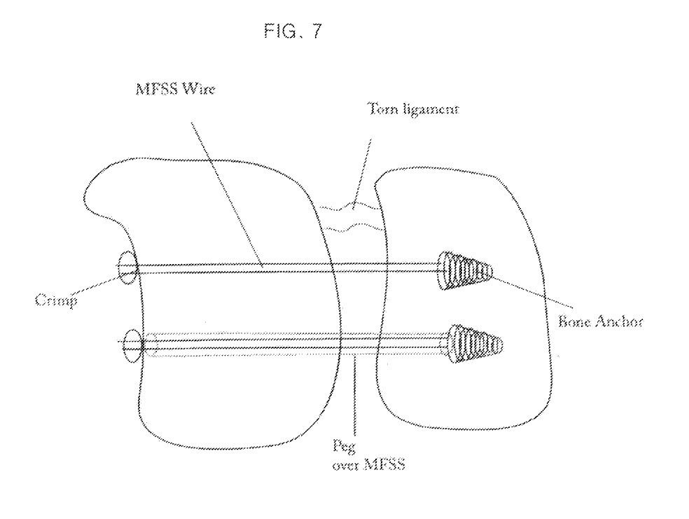

FIG. 7 illustrates a repair of the present invention similar to FIG. 6 but using a combination of crimps and anchors. A repair of this nature is useful when the bone to the right is a part of a joint. In that case, it is preferred that anchors are used so that crimps are not placed into a joint. This also simplifies the surgery since the tissues of the joint do not need to be disturbed to effect the repair. In this figure, the lower stabilizer is MFSS inside an anchor or cannulated peg. Alternative stabilizers could be just a peg, a MFSS cable or other stabilizer as desired.

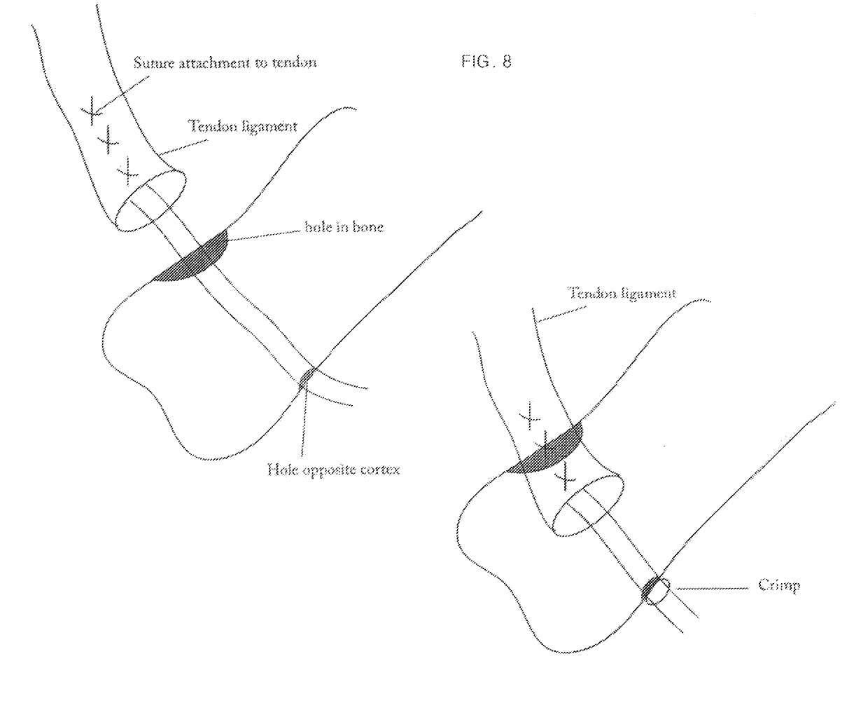

FIG. 8 illustrates a repair method of the present invention wherein a tendon or ligament is repaired. A wire suture, preferably a multifilament wire suture is stitched into the tendon or ligament. A passage is drilled through the bone for the passage of the wire. The opening of the passage where the tendon will heal to the bone is over drilled to accommodate the end of the tendon. The wire is then passed through the passage, tension is applied to pull the tendon or ligament into the passage and a crimp is applied to hold the wire in place.

This type of repair is particularly advantageous for the repair tendons torn off of bone by strong muscles. For example, a torn Bicep tendon. The advantage is in the use of the wire and crimp which provide exceptionally good strength to hold the tendon in place during the healing process.

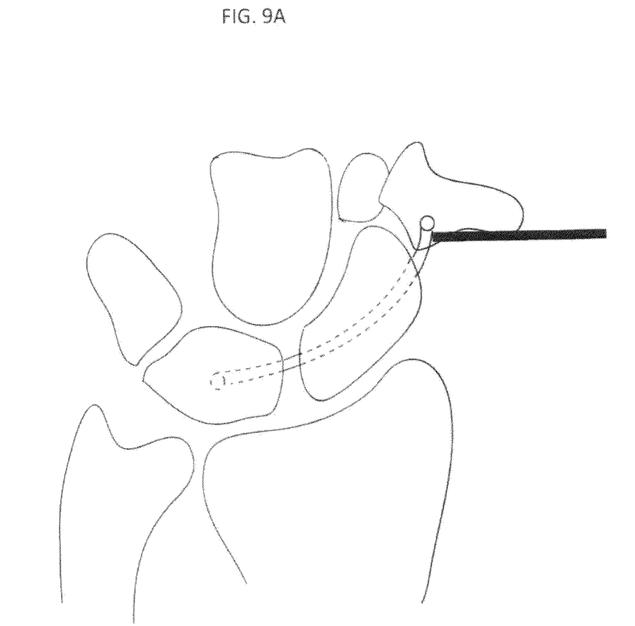

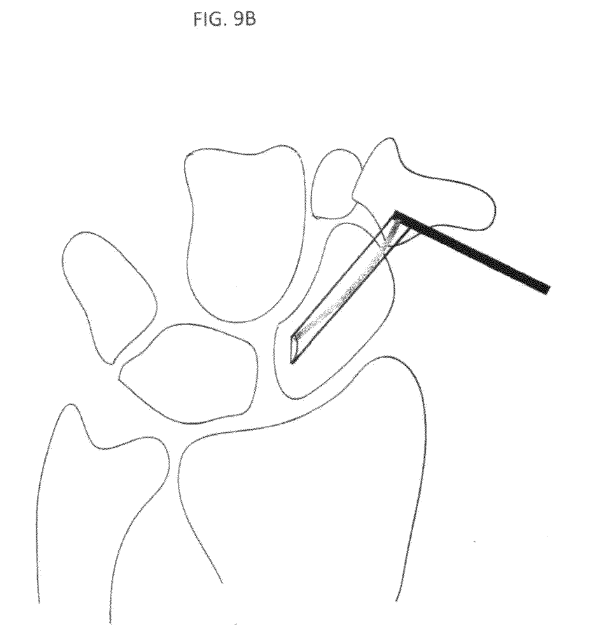

The present invention is particularly useful for repairing injuries to the Scapho-Lunate ligament. FIG. 9A shows the bones of the wrist overlaid with a curved track where a passage will be drilled (dotted lines). A curved or straight drill guide is placed at the point where the passage will begin in the Lunate bone and end in the Scaphoid bone.

In FIG. 9B, a method of producing a curved bore for repairing a Scaphiod Lunate ligament is illustrated. Initially, a straight drill guide is used to start the drilling by creating a straight hole in the Scaphoid bone as in FIG. 9A. A second drill guide (shown in FIG. 9B) is then passed into the bore in the bone. This second guide has the same outside diameter as the first but has an internal cannulated guide that is thinner and lies within the large bore of the second drill guide. This smaller internal guide has a desired curvature. It is used to position a guide wire with the exact curvature desired for the subsequent placement of the stabilizer.

A guide wire is then passed through the smaller internal guide and the drill guide is then removed leaving the guide wire in place. A cannulated curved drill is then passed over the guide wire to drill the appropriately curved bore into the Lunate bone by drilling over the curved guide wire. Once the curved bore is formed in the bones, a curved tube is placed in the bore through the Scaphoid and into the Lunate. The guide wire is then removed. At this point a cannulated or multi-filament cable stabilizer appropriate for the desired method of fixation is then passed through the tube to complete the fixation. The distal fixation is typically accomplished using an anchor. Once the anchor is placed in the Lunate bone, the tube may be removed and the stabilizer is crimped adjacent the proximal entry point of bore in the Scaphoid bone. In some embodiments, if the tube is appropriately flexible, the tube may be left in place.

When performing this method it can be useful to incorporate radio opaque materials into the drill guides, drills, stabilizers and guide wires. The use of these materials can make it possible to use x-rays during the operation to position the devices to make the desired curved and straight bores and to verify proper placement of the stabilizer.

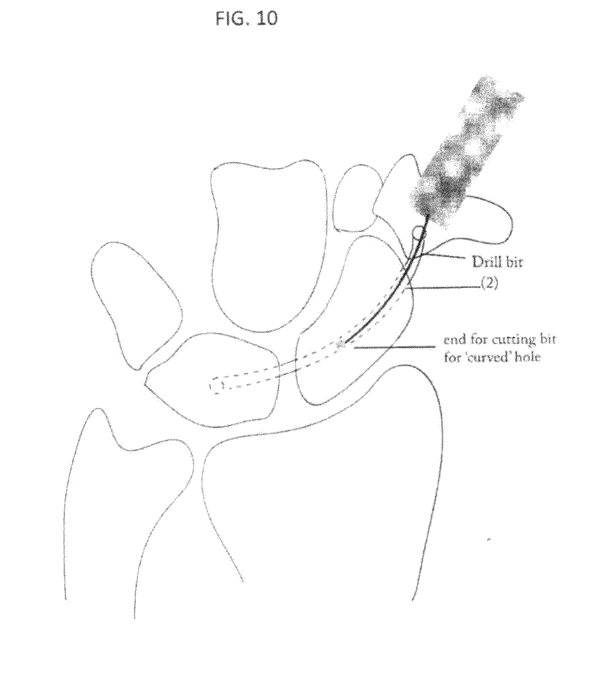

A drill bit (FIG. 10, (2)) for cutting a curved or straight passage to accommodate a MFSS wire is used to drill through the axis of the bones. This step can be performed in small increments followed by the drill guide or a drill bit with an inner canal that allows a pin or wire to curve as needed.

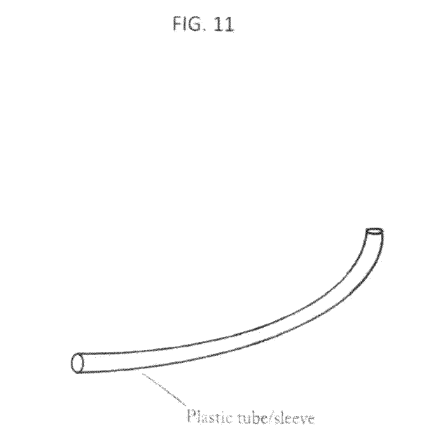

Passage of the multi filament stainless steel (or other flexible material) is done using the drill guide or by placing a tube (FIG. 11) of some other substance, i.e.: plastic, silastic or another material that can conform to the drill hole. This is placed into the drilled hole to the far end of the hole, into the drill guide or freehand. A guide pin may be used to find the initial path and the drill bit cannulated to drill over the guide wire followed by the tube placement.

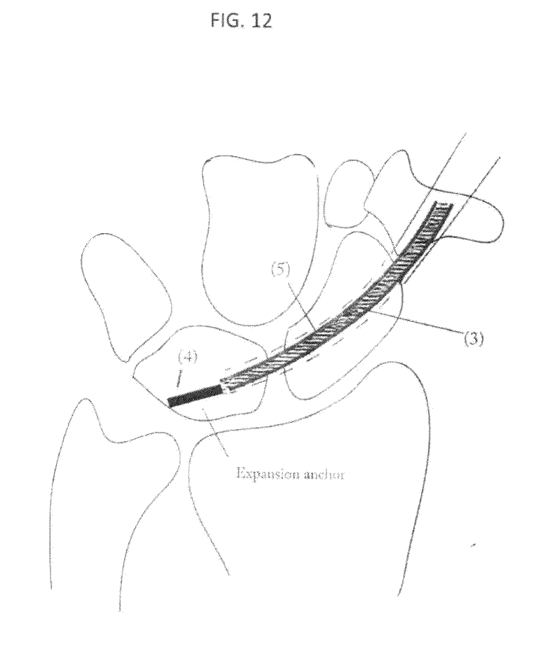

In FIG. 12, a MFSS suture with an anchor or a specially designed device on the end (expansion anchor) that will fit flat into the drill guide or the tube is placed within the tube so as to position it within the drilled hole. The coating of the cable or suture is shown. In this step, a stabilizer such as a multi filament stainless steel cable or other suture with or without specific coating can be placed within the drill guide or sleeve from the first bone to the second one and into the core of the second bone, or can be placed through the far cortex of the second bone. The anchor is attached to the far end of the stabilizer and initially lies flat within the guide or tube. It is then pushed out the far end of the tube and changes position or orientation. The change in position or orientation allows the anchor to ‘hold’ in the bone or through the far cortex of the second bone.

The MFSS can be coated or treated (5) in many ways with nylon, a polymer or any other synthetic substance or have a biologic material surrounding or embedded in it. This could also have a coating with a porous material for ingrowth of bone. The MFSS being fairly firm can be pushed into the hole as the drill guide or tube is removed leaving the MFSS in position.

The expansion device or anchor at the far end of the MFSS (or in repairs where a solid wire is used, at the far end of the solid wire) is pushed into the far bone. The anchor (4) can be a standard anchor with threads as is currently used, tubular, cylindrical, a series of balls or have spikes or be of any variable shape. It may lie initially within the tube and then expand or change orientation as it is pushed into the far hole but in any case will hold secure in the far bone. It is connected to the MFSS or the solid wire and may screw into the wire before placement or can be screwed on after the wire has been passed through the proximal bone and before it is passed into the distal bone.

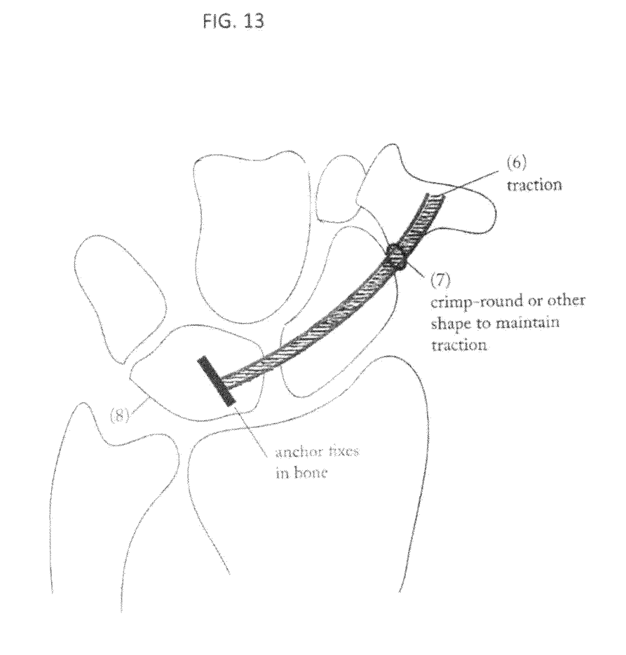

Once anchored in the far bone e.g.: Lunate bone, the MFSS passes out of the near bone e.g.: Scaphoid bone and traction applied thereby bringing the bone segments closer and compressing the space between the bones. See FIG. 13. The Scaphoid bone may contain the anchor and the Lunate the crimp. A crimp as described above is employed to fix the MFSS at the near exit fixing it in position and sustaining fixed compression to hold the two bones together. The distal end of the MFSS is secured in the far bone by an anchor and the proximal end is secured by the crimp.

When using the present method to repair a Scaphiod-Lunate ligament, it is preferred that a flexible stabilizer be used. A flexible stabilizer such as a multifilament stainless steel cable allows for rotational movement between he Scaphoid and Lunate bones resulting in a more natural movement of the wrist than is possible using solid wires or screws. The amount of movement allowed by the cable can be delimited by the design of the cable, i.e., how stiff it is made to be. It is also useful to note the lay with which the cable is manufactured as it will influence the direction in which the cable can rotate. Cables of opposite lay would be appropriate for repair of Scaphoid-Lunate ligaments of the left and right hand.

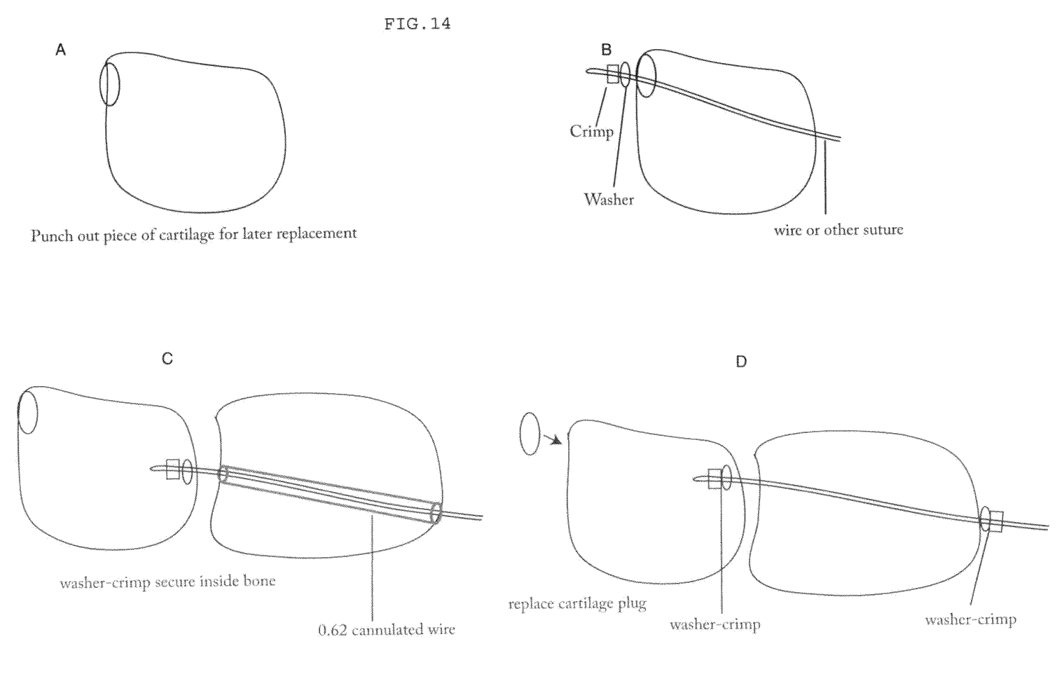

Another description of repairing the Scapho-Lunate is as follows. A small window of articular cartilage is punched out of the Lunate to prepare for the cable, a crimp and optionally a washer (FIG. 14A).

A hole is drilled across the Lunate and out the articular surface on the far side of the Lunate for passage of the cable into the region of the Scapho-Lunate joint (FIG. 14B). Alternatively the drill hole may be brought from the articular surface of the Lunate adjacent to the Scaphoid.

A cannulated wire is placed through this drilled hole. The multi filament stainless steel cable is passed through this drilled hole by passing it through the cannulated wire. The cannulated wire is then removed. A crimp and optionally a washer is placed on the cable as shown in FIG. 14B. This crimp and washer or similar implant acts to prevent the cable from pulling out of the Lunate.

A hole is drilled with a cannulated wire from the non articular or other radial part of the Scaphoid towards the Lunate. Alternatively, the hole may be drilled in the opposite direction from the Lunate articulation to the radial surface of the Scaphoid. This hole may be drilled using a cannulated wire with an optional insert inside the cannulated wire (e.g. a regular Kirschner wire) for added strength with subsequent removal of the wire insert (trocar) leaving the cannulated wire in place. The multi filament stainless steel cable is then passed from the Lunate through the cannulated wire to the radial side of the Scaphoid (FIG. 14C). Once the cable is placed the cannulated wire can be removed.

The multi filament stainless steel cable is thereby positioned within the Lunate and Scaphoid and a segment of cable now connects these bones (FIG. 14C). This segment of cable holds the bones connected and in position while at the same time allowing rotation and flexion.

Traction is applied to the cable to reduce the Scapho-Lunate interval and approximate the bones. A crimp and optionally a washer is then placed on the cable on the radial side of the Scaphoid to hold the bones in position and complete the repair (FIG. 14D).

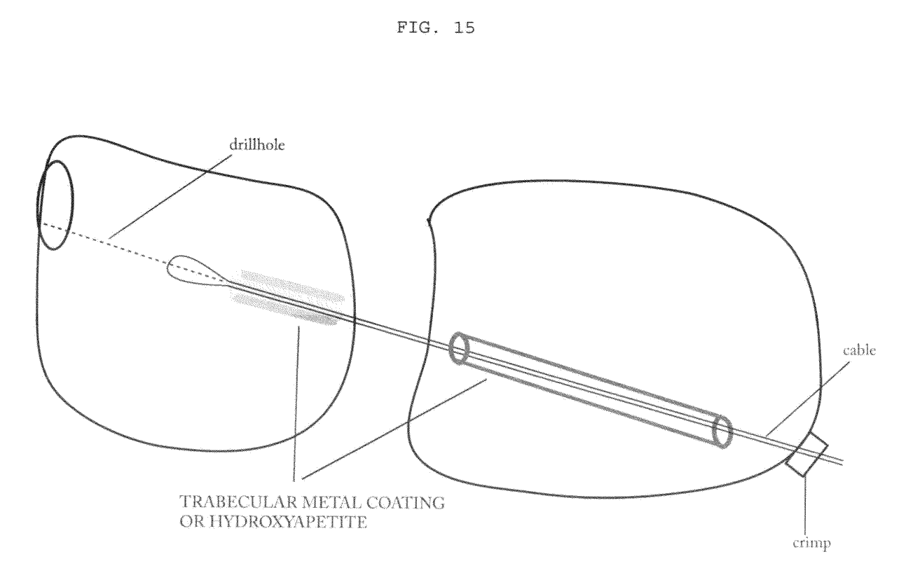

As described, in an alternative method of placement of the steel cable the cable can be inserted from the radial or ulnar side of the Scaphoid and from the radial or ulnar side of the Lunate. An anchor or cable loop may be used to hold the cable in place within the Lunate (FIG. 15) and a crimp with optional washer is placed on the far surface of the Scaphoid to hold the cable.

In another embodiment a sleeve of trabecular metal coating or hydroxyapatite or other bone ingrowth material can be positioned in the Lunate and the Scaphoid around the stainless steel cable allowing ingrowth of bone or tissue into the coating to hold the cable in position within the bones (FIG. 15).

Two bones can be approximated and fixed in a reduced position using cable, crimp and bone sleeves or anchors. This may be used for any two or more bones such as in the shoulder, foot, ankle, wrist or other areas. One example would be the reduction of a scapho-lunate disruption in the wrist.

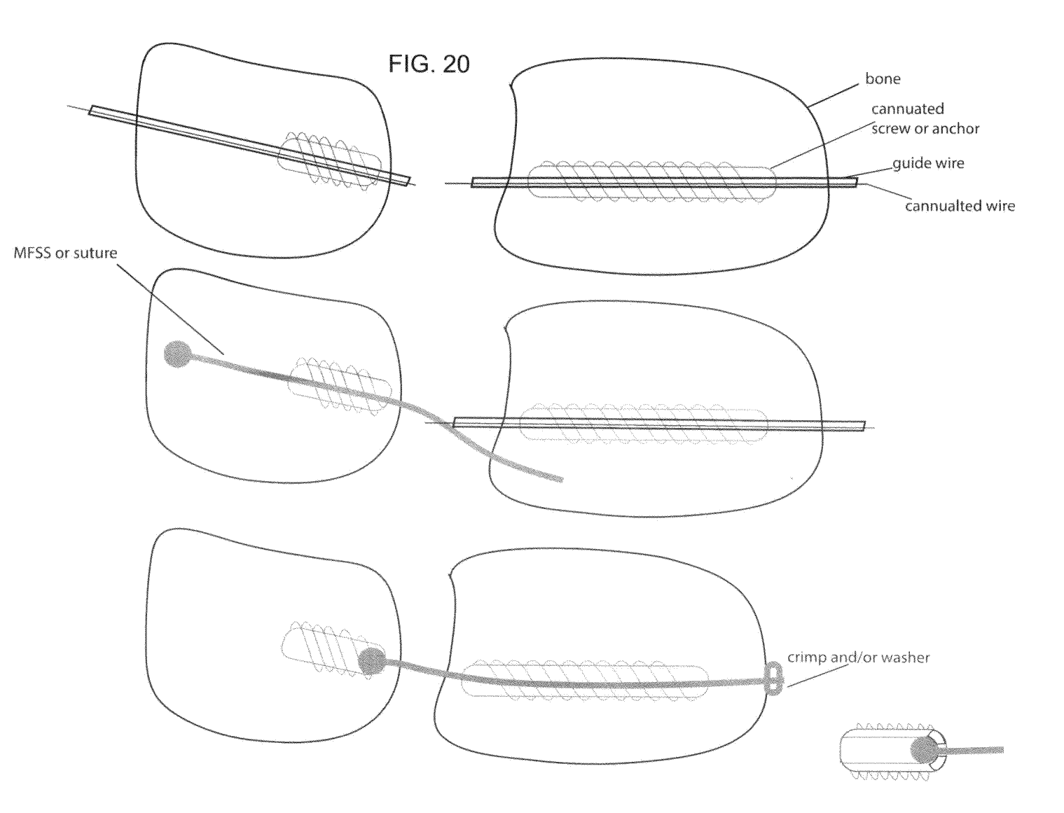

The sleeve or metal coating (FIG. 20) may take the form of a cannulated headless screw or anchor placed within the bone and the suture or a multi filament stainless steel cable is passed through this metal sleeve or anchor.

Various methods may be used to pass the suture or cable through the sleeve or anchor. For example, a guide-wire can be used for initial positioning of the sleeve or anchor. A cannulated wire (0.062 inch or other sizes) can then be placed over the guide wire and the sleeve or anchor may then be positioned over the cannulated wire before being screwed or placed in the bone. The guide-wire can then be removed and the multi filament stainless steel cable or suture passed through the cannulated wire. The cable or suture may optionally have an anchor on one end such as the ball shown in FIG. 20. One advantage of a ball shaped anchor on the cable or suture is that it can rotate inside an appropriately shaped sleeve or anchor. The rotation can reduce any torsional stress placed on the cable or suture. The cannulated wire is removed so that the suture or cable will rest within the cannulated sleeve or anchor. On the side where the suture or cable has the expanded ball it is necessary to remove the cannulated wire before passing the cable. (FIG. 20)

In the second bone the sleeve or anchor is placed in a similar fashion and a guide-wire and cannulated wire may be used as a guide to position the sleeve or anchor.(as had been done in the first bone)

The cable that emanated out of the first bone is then passed through the cannulated wire that comes out of the second bone. This cannulated wire lies within the sleeve or anchor. Once the cable is passed through the cannulated wire and out of the far end of the second bone then the cannulated wire is removed leaving the suture or cable within the sleeve or anchor.

With traction on the cable the ball is caught within the anchor which lies within the first bone causing a force on the anchor and thereby on the first bone causing traction on the bone to effect the reduction. The ball at the end of the cable may be fixed or may rotate within the anchor and thereby reduce the torque on the cable.

Once the sleeves or anchors are in position with the cables within them running between and within the two bones appropriate traction is applied and a crimp is placed on the far end of the second bone fixing the bones in the desired position (as already described herein).

The sleeve anchor or screw may be coated with hydroxyapatite or some other substance to provide a stronger bond between the implant and the bone. Also the ball and cable as described above may be coated or a coating may be placed within the sleeve, screw or anchor such as polyethylene or ceramic to reduce friction or metallosis and wear over time.

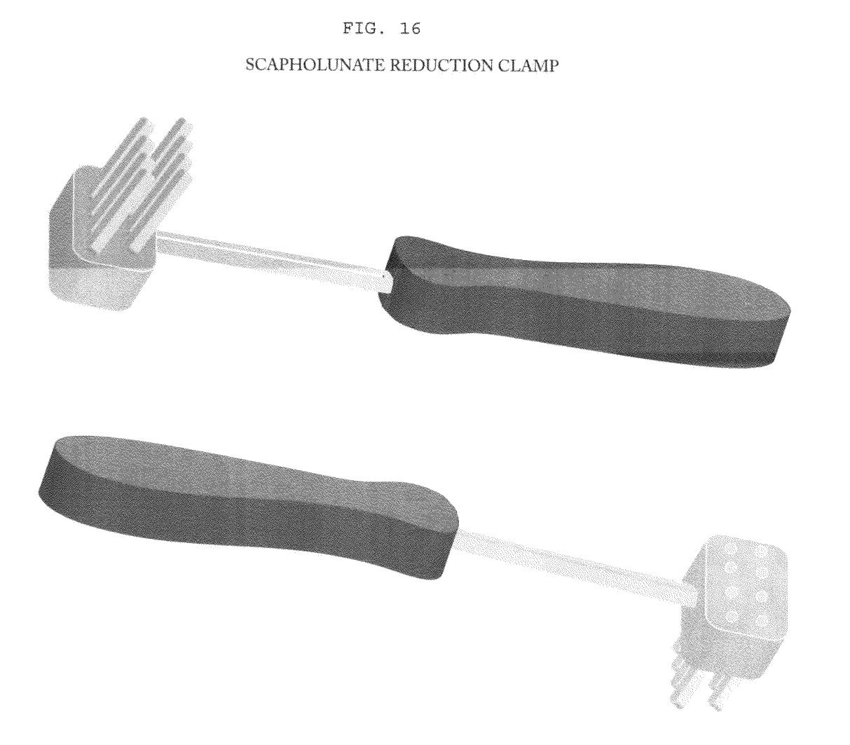

An adjunct to this procedure is a Scapho-Lunate reduction clamp (FIG. 16). A 0.062 or other size Kirschner wires are placed in the Lunate and the Scaphoid bones. The wires are brought together using them as “joy sticks” which allows the surgeon to reduce the bones and correct the position. “Joy sticks” are currently in frequent use. The Kirschner wires are placed in the appropriate holes in the reduction clamp and this allows the Scapho-Lunate reduction clamp to hold the two bones together and in the correct rotation (usually flexing the Lunate and extending the Scaphoid) while the cable is placed as described above. This allows the correct position to be maintained while the fixation as described above is placed.

The methods of the current invention can be applied to the repair of the Achilles tendon and other large tendons as illustrated in the following description.

A small transverse incision is made possible by the simple method of tendon attachment and the strength of the steel cable and crimp. Once the cable is attached to the tendon on both sides, the two tendon ends are brought together which can be accomplished under an intact skin bridge. The cables are then attached together using a crimp which can also be done through a small transverse incision. Therefore the repair can be performed through small incisions while maintaining tension and strength on the repair. The cable-crimp repair is strong and simple to perform. The method has not previously been described to repair large soft tissues such as the Achilles tendons.

In situations where the tendon is avulsed from the Calcaneus bone, the cable can be used to attach the tendon to the Calcaneus utilizing a crimp on the far end of the bone instead of using a suture anchor. This is a simpler and stronger method of bone attachment for this tendon compared with using a bone anchor.

The method is described below in three steps, the incision, the tendon attachment and the placement of the crimp. Thereafter an embodiment is described for situations where the tendon is avulsed from the Calcaneus. A similar method can be used to repair or attach other large tendons and the method is not limited to the Achilles tendon.

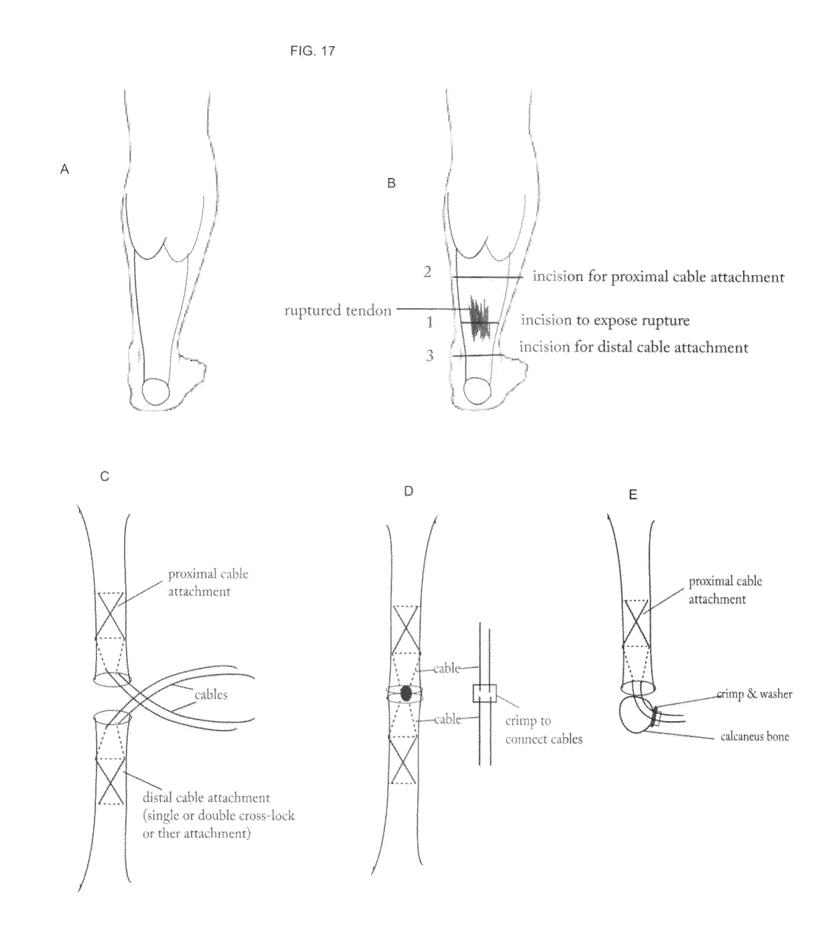

In FIG. 17B, an incision is made at the site of the tendon rupture. This may be a small transverse incision to expose the tendon damage and to confirm the site for the later connection with the crimp. Small back cuts can be made to provide slightly greater tendon visualization.

A second incision is made proximal to the first. This is also a limited transverse incision and made at least as far from the first incision so that the width of the first incision is half of the distance between the first and second incisions.

A third incision is made distal to the first incision. This is also transverse and also the same distance from the first incision.

Each of these incisions may have a small back cut of 2 to 5 mm to provide slightly better exposure of the proximal and distal intact tendon.

The purpose of the second and third incisions is to provide access to undamaged portions of the tendon on each side of the site of damage. The undamaged portions of the tendon are then accessed for attachment of the cables used in the repair.

A cross-lock attachment is made to secure the cable to the Achilles tendon through the small transverse incision (FIG. 17C). Multi filament stainless steel may be used or other suture materials may be used. The suture may be coated or lined to provide a better surface for gliding or attachment to the tendon or ingrowth of cells or to promote healing. The cable-crimp ultimate tensile strength is 300N to 500N depending on the cable and crimp size. The attachment to the tendon of the cross lock is similar strength and depends on the quality of the native undamaged tendon. Any configuration of attachment may be used. A cross lock attachment is described in PCT application PCT/US12/041063. One two or more cross locks may be used.

The sutures are passes beneath the skin from the proximal (2 in FIG. 17B) and distal (3 in FIG. 17B) incisions to the first incision (1 in FIG. 17B) above. This passage may be by passing a cannulated wire from the proximal incision or distal incision to the first incision and passing the cables through the cannulated wire. Other methods of passing the wires may be used, such as a Nitinol loop or clamp.

Through the first incision the cables are retrieved and passed through the crimp. The crimp is the same metal as the cables in the case of stainless steel but may be of other materials if other sutures are used. A requirement of the material used is that the crimp can be deformed to trap the cables thus holding the proximal and distal portions of the tendon together. Traction is then applied to each cable in order to approximate the tendon ends. A crimp tool is used to compress the crimp and thereby connect the tendon ends (FIG. 17D).

If the Achilles tendon has been avulsed from the Calcaneus then the tendon end can be attached to the bone directly. The cable is attached to the avulsed tendon as described in the method above. The tendon and cables are then attached to the calcaneus bone by drilling through the bone and bringing the cable ends out through the bone.

Traction is applied to the cables and a crimp with optional washer is then placed on the far side of the bone to secure the cable in position and hold the tendon in position. A washer may be placed between the bone and the crimp prior to crimping. In certain embodiments, the crimp and washer may be recessed into the bone.

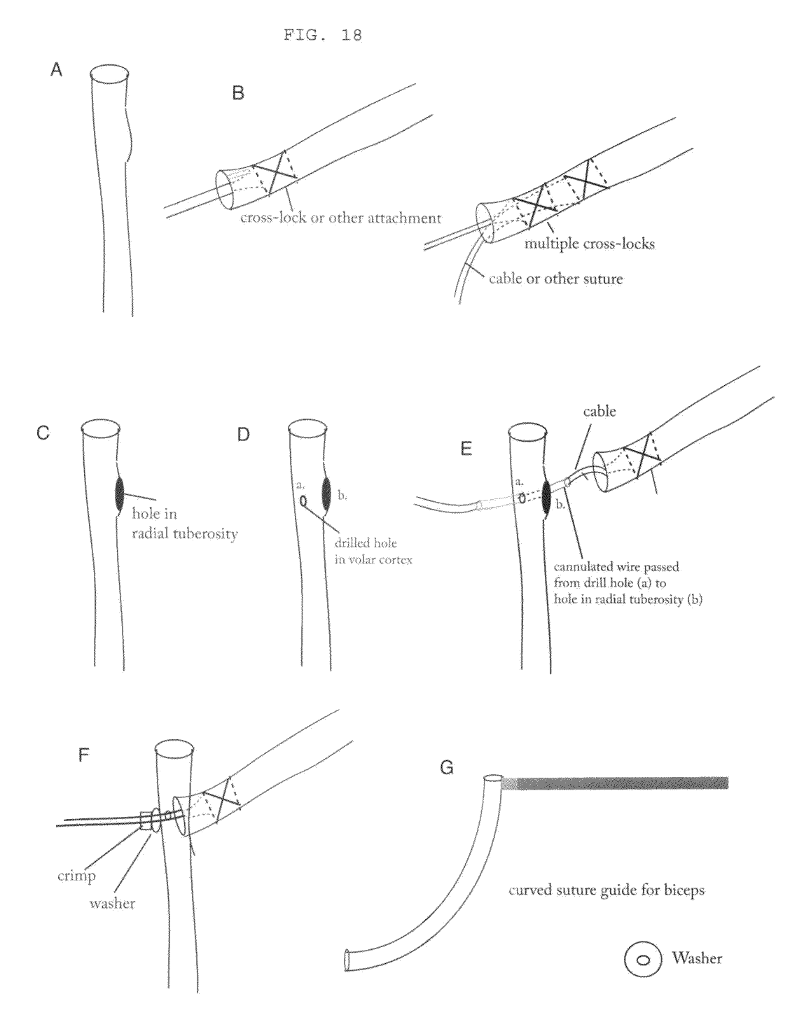

The present invention can be applied to the repair of the Biceps tendon, particularly avulsions of the Biceps tendon from the radial tuberosity. An exemplary method is performed as follows. The first step is to identify and dissect the distal avulsed end of the Biceps tendon. A cross lock stitch is used to place the cable in the cut end of the tendon (FIG. 18B). Other methods of attachment may also be used. (The method of the cross-lock attachment is described in PCT application PCT/US12/041063.) One, two or more cross lock stitches may be used to attach the cable to the tendon. Multi filament stainless steel cable is used for this repair. Other sutures such as Fiber Wire, Power Fiber, Force Fiber or others may be used. The Multi filament stainless steel may be coated for gliding or to deliver growth factors or other substances used to promote healing or improve attachment.

The surgeon roughens the radial tuberosity or drills a hole in the radial tuberosity for acceptance of the cut end of the tendon (FIG. 18C). A hole is drilled in the volar cortex of the radius at a point that leaves a large enough bridge of bone between the radial tuberosity to prevent the cable pulling through (FIG. 18D).

The cables that are attached to the cut end of the tendon are passed through the radial tuberosity and brought out through the volar drilled hole. A cannulated guide (FIG. 18E) can be used to facilitate the passages of the cables, In this case the cannulated guide (FIG. 18G) is passed through the hole in the radius and out the radial tuberosity and the cables are then passed through the cannulated guide. The cannulated wire or guide is then removed. Other methods of passing this cable, e.g., a Nitinol suture basket or sling may be used.

Traction is then applied to the cable pulling the tendon into the radial tuberosity or firmly against it. Tension is maintained while a washer and crimp are applied to the cable thereby holding it firmly in position (FIG. 18F). In certain embodiments the washer and crimp may be recessed into the surface of the bone.

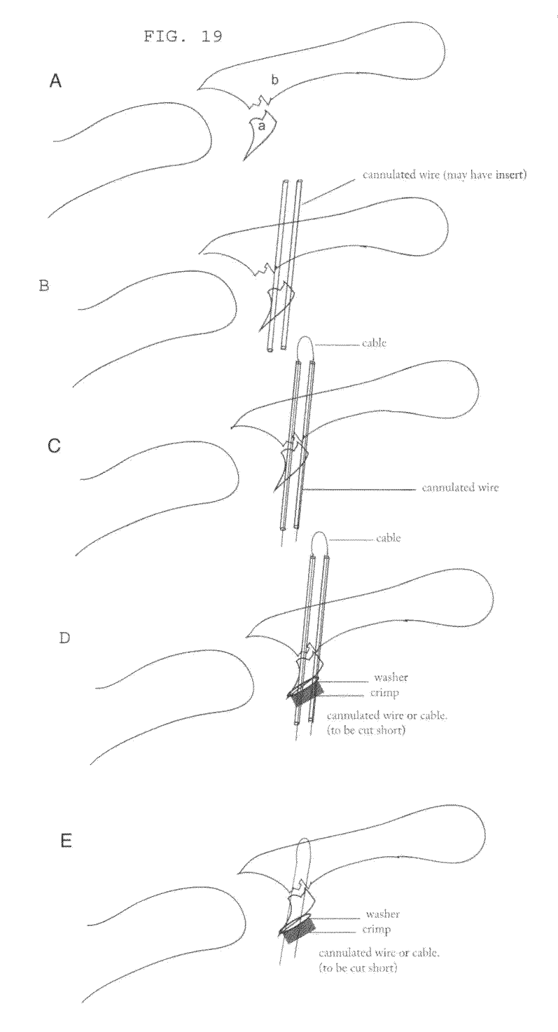

Methods of the present invention can be applied to the repair of fractures in the Proximal Interphalageal (PIP) joint. In one example, the current invention can be applied to re-locate the dorsal fragment (“b” in FIG. 19A) and fix it to the volar fragment (“a” in FIG. 19A). The following method is described as an example.

A hole is drilled in the volar fragment and then into the dorsal bone. This can be done with a cannulated wire drilled through the volar fragment, into the dorsal bone and through dorsal cortex. For strength a Kirschner wire may be inserted inside the cannulated wire (as a trocar) to prevent breakage (FIG. 19B).

The Kirschner wire trocar is removed leaving the cannulated wire in place. Multi filament stainless steel cable (or other sutures) is then passed through the cannulated wires to form a loop around the dorsal bone (FIG. 19C).

A crimp is used to crimp the wire cable on the volar side of the volar fragment thereby holding the dorsal and volar fragments apposed and in position and preventing dislocation as traction is applied. A washer may be employed ahead of the crimp (FIG. 19D). Traction is applied to the cannulated wire to reduce the dorsal fragment and bring it in close proximity to the volar fragment and volar plate.

A crimp, and optionally a washer, is then applied to the volar fragment thereby holding the fracture in position and correcting the dislocation (FIG. 19D and 19E).

Although a particular repair is described above, other combinations of the above stabilizers, singly or multiply, with crimp(s) and anchor configurations are possible.

Even though description of the utility of the various embodiments was limited to the certain bones, tendons and ligaments, it must be understood that many bone, tendon and ligament repairs can be carried out by use of the methods and devices as described, either in part of in full. Examples of such anatomical structures include the bone, tendons and ligaments of the body as well as any other structure require fixation in single or multiple points.

An exemplary method of repairing or connecting a bone fracture or other separation comprises drilling at least one passage through a bone having a separation, the passage having a first and second opening and the passage crossing the separation, positioning a deformable stabilizer in the passage, the stabilizer having a first end and a second end and being longer than the passage, fixing the first end of the stabilizer at an opening of the passage, applying traction or distraction to the second end of the stabilizer to apply compression or distraction to the separation, and fixing the second end of the stabilizer at the other opening of the passage by applying a crimp to the second end of the stabilizer, said crimp deforming the stabilizer.

This method can include having the first end of the stabilizer is fixed at an opening of the passage by a technique selected from the group consisting of applying a crimp to the first end of the stabilizer, forming a capped end on the first end of the stabilizer, applying an anchor to first end of the stabilizer, placing a screw in the opening of the passage to fix the first end of the stabilizer, and then fixing the second end of the stabilizer by applying a crimp to the second end of the stabilizer.

The method can be used where the stabilizer is a cannulated wire and the anchor is an umbrella anchor.

The method can be used where the passage is a curved passage and the stabilizer is a flexible wire or cable.

The method can be used where the stabilizer is a multifilament stainless steel wire.

The method can be used where in the stabilizer is cannulated, the stabilizer is fixed in place with a crimp and the crimp deforms the stabilizer.

The method can be used where the bones may be separated by a fracture in one or multiple parts or an osteotomy made by cutting the bone.

The method can be used where the stabilizer is a rigid wire.

The method can include the use of a perforated plate to hold the fracture or separation in place in conjunction with at least one stabilizer.

The method can include the use of a reduction plate that is used for temporary correction of longitudinal or rotational bone position and is removed after fixation.

Another exemplary method of repairing a bone separation can include drilling at least one first passage through a first bone fragment and at least one second passage into a second bone fragment, the first and second passages being aligned with each other linearly or along an arc across the fracture, positioning a stabilizer having a first end and a second end through the first passage and into the second passage, the stabilizer being longer than the distance through the two passages, fixing the first end of the stabilizer in the second passage, applying traction to the second end of the stabilizer to position the first and second bone fragments in a desired proximity to each other and compressing them together, and fixing the second end of the stabilizer at the opening of the first passage by applying a crimp to the second end of the stabilizer.

The method can be used where the first end of the stabilizer is fixed in the second passage by an anchor at the first end of the stabilizer.

The method can be used where the passage is a curved passage and the stabilizer is a flexible wire or cable.

The method can be used where the stabilizer is a multifilament stainless steel wire.

The method can be used where the crimp is countersunk into the cortex of the bone.

The method can include the use of a perforated plate to hold the fracture or separation in place in conjunction with at least one stabilizer.

A method of repairing a ligament between two bones can include drilling at least one first passage through a first bone and at least one second passage through a second bone, the first and second passages being linearly aligned with each other and approximately parallel to the direction of the ligament to be repaired, positioning a stabilizer having a first end and a second end through both the first and second passages, the stabilizer being longer than the distance through the two passages, fixing the first end of the stabilizer at the farthest opening the second passage, pulling the second end of the stabilizer to position the first and second bones in a desired proximity to each other, and fixing the second end of the stabilizer at the farthest opening of the first passage by applying a crimp to the second end of the stabilizer.

The method can be used where the passage is a curved passage and the stabilizer is a flexible cable.

The method can be used where the stabilizer is a multifilament stainless steel wire.

The method can be used where the first and second bones are a Scaphoid and a Lunate bone and the ligament to be repaired is the Scapho-Lunate ligament.

The method can be used where the stabilizer passes through the ligament.

The method can be used a cannulated anchor is placed in the second passage in the bone, an anchor is attached to the first end of the stabilizer and the anchor fixes the stabilizer in the cannulated anchor. The method can be used where the anchor attached to the stabilizer is a ball and the ball is able to rotate within the cannulated anchor.

A method of repairing a ligament between two bones can include drilling at least one first passage through a first bone and at least one second passage into a second bone, the first and second passages being linearly aligned with each other and approximately parallel to the direction of the ligament to be repaired, positioning a stabilizer having a first end and a second end through the first passage and into the second passage, the stabilizer being longer than the distance through the two passages, fixing the first end of the stabilizer in the second passage, pulling the second end of the stabilizer to position the first and second bones in a desired proximity to each other, and fixing the second end of the stabilizer at the farthest opening of the first passage by applying a crimp to the second end of the stabilizer.

The method can be used where the passage is a curved passage and the stabilizer is flexible.

The method can be used where the stabilizer is a multifilament stainless steel wire.

The method can be used where the first and second bones are a Scaphoid and a Lunate bone and the ligament to be repaired is the Scapho-Lunate ligament.

The method can be used where the first end of the stabilizer is fixed in the second passage by an anchor at the first end of the stabilizer.

A method of connecting a tendon to a bone can include attaching a stabilizer to the end of a tendon, drilling a passage through the bone, passing the stabilizer through the passage to the opposite side of the bone, securing the stabilizer with a crimp placed on the stabilizer outside the opposite side of the bone to secure the stabilizer and hold the tendon to the bone.

The method can be used where the stabilizer is cannulated and the application of the crimp deforms the stabilizer.

A method of creating a curved bore can include drilling a straight bore into a bone, inserting a first cannulated drill guide into the straight bore, inserting a second curved cannulated drill guide into the first cannulated drill guide, using the second drill guide to position a curved guide wire, removing the second drill guide and positioning a curved cannulated drill over the curved wire, drilling a curved bore with the curved drill and removing the curved drill, inserting a tube into the straight and curved bores,

removing the guide wire and inserting a stabilizer with an anchor into the tube, anchoring the stabilizer in the bone at the end of the curved bore, and crimping the stabilizer adjacent the beginning of the straight bore.

The method can be used where the straight bore is drilled in a Scaphoid bone, the curved bore is drilled beginning in the Scaphoid bone and ending in the Lunate bone, and the anchor on the stabilizer is placed in the Lunate bone.

A useful stabilizer of this invention is a deformable stabilizer with predetermined weakened points.

A useful stabilizer of this invention is a cannulated stabilizer with predetermined weakened points.

A useful stabilizer of this invention has a first end and a second end, the first end having a larger diameter than the second end.

Having thus described particular embodiments of the invention, various alterations, modifications, combinations of application and improvements will readily occur to those skilled in the art. Such alterations, modifications, combinations and improvements as are made obvious by this disclosure are intended to be part of this description though not expressly stated herein, and are intended to be within the spirit and scope of the invention. Accordingly, the foregoing description is by way of example only, and not limiting. The invention is limited only as defined in the following claims and equivalents thereto.

1. A method of repairing a separation comprising:
drilling at least one passage through a bone having a separation, said passage having a first and second opening and said passage crossing said separation, positioning a deformable stabilizer in said passage, said stabilizer having a first end and a second end and being longer than said passage, fixing the first end of the stabilizer at an opening of said passage, applying traction or distraction to the second end of the stabilizer to apply compression or distraction to achieve the desired proximity across the separation, and fixing the second end of the stabilizer at the other opening of said passage by applying a crimp to the second end of the stabilizer, said crimp deforming the stabilizer.

2. The method of claim 1 wherein said first end of the stabilizer is fixed at an opening of the passage by a technique selected from the group consisting of applying a crimp to the first end of the stabilizer, forming a capped end on the first end of the stabilizer, applying an anchor to first end of the stabilizer, placing a screw in the opening of the passage to fix the first end of the stabilizer, and then fixing the second end of the stabilizer by applying a crimp to the second end of the stabilizer.

3. The method of claim 1 wherein said passage is a curved passage and said stabilizer is a flexible wire or cable.

4. The method of claim 1 in which the bones may be separated by a fracture in one or multiple parts or an osteotomy made by cutting the bone.

5. The method of claim 1 wherein the stabilizer is a rigid wire.

6. The method of claim 1 further comprising the use of a perforated plate to hold the fracture or separation in place in conjunction with at least one stabilizer.

7. A method of repairing a bone separation comprising:
drilling at least one first passage through a first bone fragment and at least one second passage into a second bone fragment, said first and second passages being aligned with each other linearly or along an arc across the fracture, positioning a stabilizer having a first end and a second end through the first passage and into the second passage, said stabilizer being longer than the distance through the two passages, fixing the first end of the stabilizer in the second passage, applying traction to the second end of the stabilizer to position the first and second bone fragments in a desired proximity to each other and compressing them together, and fixing the second end of the stabilizer at the opening of the first passage by applying a crimp to the second end of the stabilizer.

8. The method of claim 7 wherein said first end of the stabilizer is fixed in the second passage by an anchor at the first end of the stabilizer.

9. The method of claim 7 wherein said passage is a curved passage and said stabilizer is a flexible wire or cable.

10. The method of claim 7 further comprising the use of a perforated plate to hold the fracture or separation in place in conjunction with at least one stabilizer.

11. A method of repairing a ligament between two bones comprising:
drilling at least one first passage through a first bone and at least one second passage through a second bone, said first and second passages being linearly aligned with each other and approximately parallel to the direction of the ligament to be repaired, positioning a stabilizer having a first end and a second end through both the first and second passages, said stabilizer being longer than the distance through the two passages, fixing the first end of the stabilizer at the farthest opening the second passage, pulling the second end of the stabilizer to position the first and second bones in a desired proximity to each other, and fixing the second end of the stabilizer at the farthest opening of the first passage by applying a crimp to the second end of the stabilizer.

12. The method of claim 11 wherein said passage is a curved passage and said stabilizer is a flexible cable.

13. The method of claim 11 wherein the second passage is drilled partially into the second bone, fixing the first end of the stabilizer in the second passage, pulling the second end of the stabilizer to position the first and second bones in a desired proximity to each other, and fixing the second end of the stabilizer at the farthest opening of the first passage by applying a crimp to the second end of the stabilizer.

14. The method of claim 13 wherein the stabilizer is a multifilament stainless steel wire.

15. The method of claim 13 wherein the first end of the stabilizer is fixed in the second passage by an anchor at the first end of the stabilizer.

16. A method of connecting a tendon to a bone comprising:
attaching a stabilizer to the end of a tendon;
drilling a passage through the bone,
passing the stabilizer through the passage to the opposite side of the bone,
securing the stabilizer with a crimp placed on the stabilizer outside the opposite side of the bone to secure the stabilizer and hold the tendon to the bone.

17. The method of claim 1 wherein the stabilizer is cannulated and the application of the crimp deforms the stabilizer.

18. The method of claim 1 further comprising the use of a reduction plate that is used for temporary correction of longitudinal or rotational bone position and is removed after fixation.

19. The method of claim 13 wherein a solid guide wire is used to create the first passage and the second passage, a cannulated wire is placed over the solid wire and the solid wire is removed, a cannulated anchor is placed in the second passage in the bone, the stabilizer is inserted into the cannulated wire and the first end of the stabilizer and said anchor fixes the stabilizer in the cannulated anchor.

20. The method of claim 19 wherein a ball is attached to the first end of the stabilizer, said ball fixes the stabilizer within the cannulated anchor and the ball is able to rotate within the cannulated anchor.
